(19) United States
(12) Patent Application Publication
(10) Pub. No.: US 2013/0294972 A1
(43) Pub. Date: Nov. 7, 2013

(54) ZERO-MODE WAVEGUIDE FOR SINGLE BIOMOLECULE FLUORESCENCE IMAGING

(71) Applicant: THE TRUSTEES OF COLUMBIA UNIVERISTY IN THE CITY OF NEW YORK, New York, NY (US)

(72) Inventors: Colin Kinz-Thompson, New York, NY (US); Ruben L. Gonzalez, JR., New York, NY (US); James C. Hone, New York, NY (US); Matteo Palma, New York, NY (US); Alexander Alexeevich Godarenko, Springfield, VA (US); Daniel Alexandre Chenet, New York, NY (US); Shalom J. Wind, White Plains, NY (US)

(21) Appl. No.: 13/655,947

(22) Filed: Oct. 19, 2012

Related U.S. Application Data
(60) Provisional application No. 61/554,305, filed on Nov. 1, 2011.

(51) Int. Cl. G01N 33/53

(52) U.S. Cl. CPC G01N 33/5306
USPC 422/69; 430/296; 427/163.2

(57) ABSTRACT

The disclosed subject matter provides a zero-mode waveguide (ZMW) including a substrate and at least one nano-well thereon and having a bottom surface and a side wall comprising gold. A surface of the side wall is passivated with a first functional molecule comprising polyethylene glycol. The bottom surface of the nano-well can be functionalized with at least one second molecule comprising polyethylene glycol, for example, a silane-PEG molecule. The second molecule can further include a moiety, such as biotin, which is capable of binding a target biomolecule, which in turn can bind to a biomolecule of interest for single molecule fluorescence imaging analysis. Fabrication techniques of the ZMW are also provided.

### CROSS REFERENCES TO RELATED APPLICATIONS

This application claims priority from U.S. Provisional Application No. 61/554,305, filed Nov. 1, 2011, the disclosure of which is incorporated herein in its entirety.

### STATEMENT REGARDING FEDERALLY FUNDED RESEARCH

This invention was made with government support under Grant No. ACS-RSG-09-053-01-0, awarded by the American Cancer Society. The government has certain rights in this invention.

### BACKGROUND

Single-molecule analytical methods can provide insight into biomolecular dynamics, including by extracting characteristics of molecular interactions in complex mixtures where such information could otherwise be lost in ensemble averaging.

Zero-mode waveguides (ZMWs) can include arrays of sub-wavelength apertures in a metal film that allow for the observation of single-molecule phenomena. When light is shone through a zero-mode waveguide, photons having wavelengths greater than a threshold value can be prevented from propagating through the waveguide. The remaining evanescent waves can exponentially decay at the glass/water interface of the ZMWs, leading to a very small detection volume near the interface, e.g., on the scale of zeptoliters. Thus, ZMWs can provide improved signal-to-noise ratio (S/N) of single-molecule fluorescence, permitting single fluorophore-labeled biomolecules to be observed in imaging buffers containing physiologically relevant, micromolar concentrations of fluorophore-labeled ligands.

However, such S/N gains of the ZMWs can be limited to certain concentrations, for example, with fluorophore-labeled nucleic acids, at concentrations of up to 1 micromolar. Above this concentration of fluorophore-labeled nucleic acids, and at even lower concentrations of fluorophore-labeled proteins, non-specific binding of the fluorophore-labeled biomolecules to the surface of the ZMW can undermine the S/N gains.

Accordingly, there is a need for ZMWs with reduced non-specific adsorption of biomolecules to allow for improved sensitivity of single-molecule fluorescence of the biomolecules at higher concentrations.

### SUMMARY

The disclosed subject matter provides a zero-mode waveguide (ZMW) and techniques for use thereof. In an exemplary embodiment, a ZMW includes a substrate and at least one nano-well on the substrate. The nano-well includes a bottom surface and, can include a side wall formed of gold. A surface of the side wall can be passivated with a layer of a first functional molecule comprising polyethylene glycol. The layer can be a self-assembled monolayer (SAM).

In some embodiments, the first functional molecule can have a thiol end group, and is coupled with the surface of the side wall surface of the nano-well with a S—Au bond. The first functional molecule can further comprise polyalkylene disposed between the polyethylene glycol and the thiol end group.

In certain embodiments, the bottom surface of the nano-well of the ZMW can be functionalized with at least one second functional molecule comprising polyethylene glycol. For example, the second functional molecule can be attached to the bottom surface via a Si—O—Si linkage. The second functional molecule can further include a moiety capable of binding with a target biomolecule. The moiety can be a biotin moiety, and the target biomolecule can be streptavidin. In some embodiments, the second functional molecule can include a mixture of (1) a molecule having a moiety capable of binding with a target biomolecule, and (2) a molecule having no moiety capable of binding with the target biomolecule.

The disclosed subject matter also provides methods for fabricating ZMWs. In an exemplary embodiment, a nano-well including a bottom surface and a gold side wall can be formed on a substrate, and a surface of the side wall can be passivated with a first functional molecule comprising polyethylene glycol. The first functional molecule can include a thiol end group.

In some embodiments, the method further includes functional zing the bottom surface of the nano-well with at least one second functional molecule comprising polyethylene glycol. The second functional molecule can include a silane end group. The at least one second molecule can also include a mixture of silane-PEG and silane-PEG-moiety, where the moiety is capable of binding with a target biomolecule. The moiety can be a biotin moiety, and in such case, the target biomolecule can be streptavidin.

### BRIEF DESCRIPTION OF THE DRAWINGS

FIG. 1 is a schematic cross-sectional view of a ZMW according to some embodiments of the disclosed subject matter.

FIGS. 2A and 2B are schematic diagrams illustrating the passivation of the side wall of a nano-well of a ZMW and the passivation of the bottom surface of a nano-well of a ZMW according to some embodiments of the disclosed subject matter.

FIGS. 3A-3D are diagrams illustrating an example fabrication procedure of a ZMW according to some embodiments of the disclosed subject matter.

FIG. 4 is a schematic cross sectional view of a test setup for passivating a gold surface using a thiol-PEG molecule.

FIG. 5 depicts fluorescence images of a fluorophore-labeled protein, RF1(Cy3,Cy5) as applied on a gold surface passivated with different concentration of thiol-PEG.

FIGS. 6A and 6B are fluorescence images of macro-sized wells with exposed silica bottom surface and gold side walls at different test and passivation conditions as shown.

FIGS. 7A-7C are images of ZMWs at different stages of an example fabrication procedure according to some embodiments of the disclosed subject matter.

FIG. 8 is a diagram of a test setup for fluorescence imaging using ZMWs of the disclosed subject matter.

FIG. 9A is a fluorescence image of a test protein RF1(Cy3,Cy5) as applied on the ZMWs of the disclosed subject matter; FIG. 9B is a fluorescent image of the RF1(Cy3,Cy5) as applied on a bulk silicon substrate as a comparison.

FIGS. 10A-10C are fluorescence intensity time traces indicating photobleaching of RF1(Cy3,Cy5) in different nano-wells of gold-based passivated ZMWs.

FIGS. 11A-11D are fluorescence intensity time traces and the corresponding signal-to-noise ratios for RF1(Cy3,Cy5) at different concentrations of RF1(Cy5) as test biomolecule in the background of the ZMWs according to the disclosed subject matter.

FIG. 12A is the fluorescence intensity time traces of FRETing RF1(Cy3,Cy5) in the ZMWs according to the disclosed subject matter; FIG. 12B is the FRET efficiency time trace of the RF1(Cy3,Cy5) molecule shown in FIG. 12A.

### DETAILED DESCRIPTION

The disclosed subject matter provides zero-mode waveguides (ZMWs) with modified surface adapted for fluorescence imaging of biomolecules, as well as the fabrication of the ZMWs and uses thereof.

In one aspect, the presently disclosed subject matter provides a zero-mode waveguide, which includes a substrate and at least one nano-well on the substrate. The nano-well includes a bottom surface and, can include a side wall formed of gold. A surface of the side wall can be passivated with a layer of a first functional molecule comprising polyethylene glycol. Further, the bottom surface of the nano-well can be functionalized with at least one second functional molecule comprising polyethylene glycol.

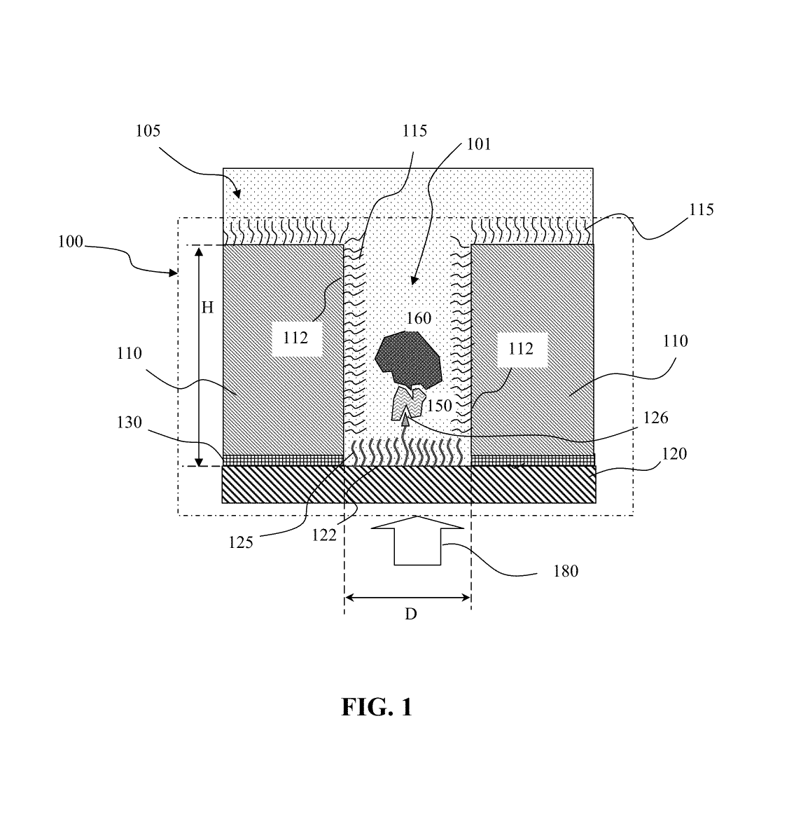

FIG. 1 is a schematic representation of the structure of a ZMW according to one embodiment of the disclosed subject matter. The ZMW includes a substrate 120, which can be a transparent material, such as glass (or silica), and a nano-well (aperture) 101 on the substrate 120. The nano-well 101 is bounded by the side wall 110 having a surface of 112. The side wall can be made from gold. An adhesion layer 130, such as a titanium layer or another metal, such as chromium, can be disposed between the underside of the side wall 110 and the substrate 120. The ZMW can be immersed in a flow cell 105, which can contain an aqueous solution of a biomolecule of interest 160, e.g., a protein, DNA, or RNA, which can include or be labeled with one or more fluorophores. These biomolecules of interest may include those which associate with other molecules, such as the ribosome, polymerases, or other enzymes, protein-binding DNA sequences, riboswitches, or ribozymes. Incident light or illumination 180 can be shone from the bottom of the substrate 120 to create a light field concentrated near the bottom of the nano-well 101.

The nano-well 101 can have a cross dimension D (width or diameter) of a few hundred nanometers, for example, 25 to 500 nm, or 200 to 250 nm, which can depend on the wavelength of the incident light used for the ZMW. The nano-well can have various cross-sectional shapes, such as circular, elliptical, multilateral, etc., as desired. A ZMW can include arrays or matrix of such nano-wells separated by the walls 110. The height (or thickness) H of the side wall 110 can be tens to a few hundred nanometers, e.g., from about 50 to about 500 nm. Smaller height can reduce the effectiveness of the ZMW as gold can be transparent at very small thickness. However, the greater the height H, the more impedance for the biomolecule of interest 160 to diffuse to the bottom of the ZMW, which can reduce the sensitivity of the ZMW. Thus, suitable height of the side wall 110 of the ZMW can be selected by balancing these considerations.

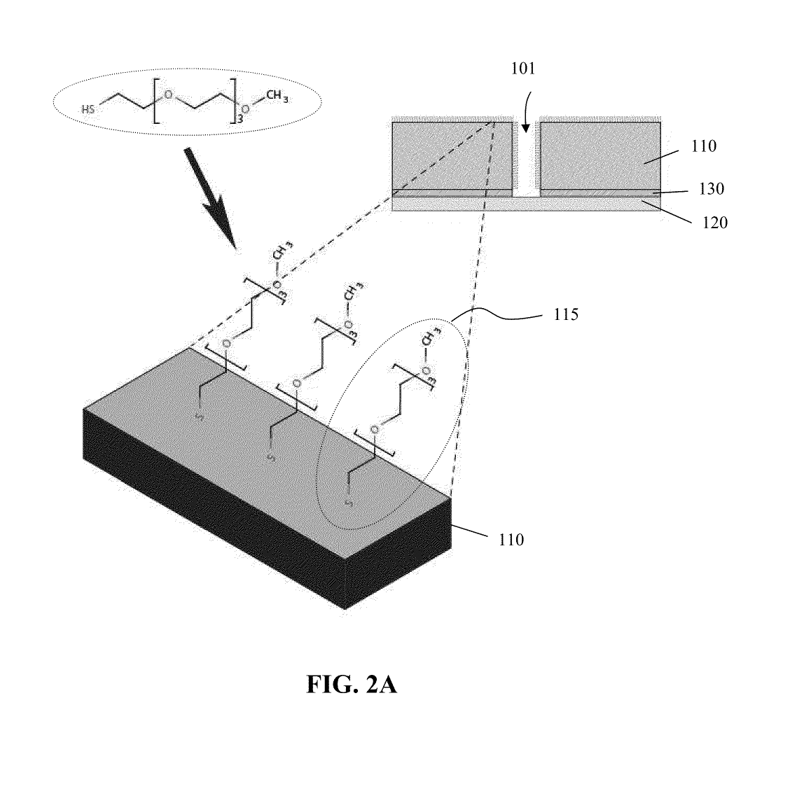

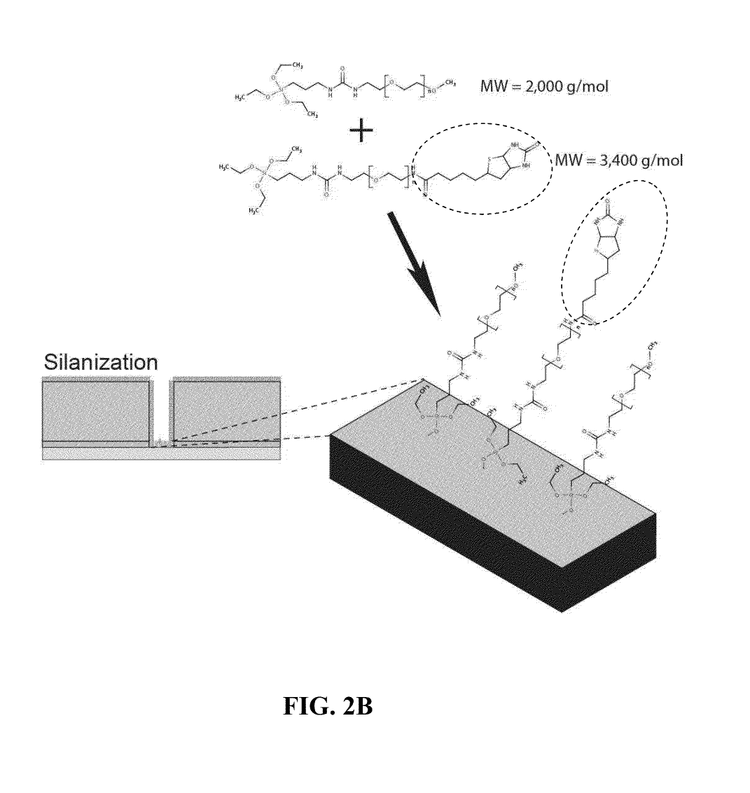

For the nano-well 101, the side wall surface 112 can be coated with a layer of a first functional molecule 115. The molecule 115 can include a segment of polyethylene glycol (PEG) to provide a non-adsorption surface to inhibit non-specific adsorption of biomolecules onto the surface 112. The PEG can include about 1 to about 200 ethylene oxide (CH₂CH₂O) units. The molecule 115 can also include a terminal thiol group, which is reactive to the gold surface of the side wall 112. Upon suitable conditions, the thiol group of molecule 115 can react with the side wall surface 112 to form S—Au bonds to couple the molecule 115 with the side wall 112. For example, the molecule 115 can form a self-assembled monolayer (SAM) tethered on the side wall 112. The molecule 115 can further include non-PEG portions, such as a segment of polyalkylene group —(CH₂)ₓ— between the polyethylene glycol and the thiol end group, where x can be from 1 to about 100, e.g., 2 to 10. Referring to FIG. 2A, which schematically illustrates thiol-passivation of the ZMW, an example functional molecule HSCH₂CH₂(OCH₂CH₂)₃OCH₃ is used to passivate the gold surface of the side wall 110 (including the top of the side wall and the surface of the side wall surrounding the nano-well 101).

Further, the bottom surface 122 of the nano-well 101 can be functionalized with a non-adsorption or passivation layer (e.g., a monolayer) of at least one second functional molecule 125 comprising polyethylene glycol. Similarly, the second molecule 125 can include about 1 to about 200 ethylene oxide (CH₂CH₂O) units. In the case where the bottom surface is glass, i.e., silica (SiO₂), the second functional molecule can include a silane end group and be attached to the bottom surface via a Si—O—Si linkage (via condensation of a silicon oxide group of the silica to the silane). The non-adsorption layer on the bottom surface 122 can further include a molecule including PEG and having a moiety 126 capable of binding with a target biomolecule 150. The binding between the moiety 126 and the target biomolecule can be based on molecular recognition or affinity, e.g., ligand-receptor type binding. In one example, the molecule can be biotinylated, i.e., include a biotin moiety. (Such a molecule is herein referred to as Biline-PEG-biotin for short). In such a case, the target biomolecule 150 can be streptavidin. Other binding moieties can also be selected, e.g. glutathione to glutathione S-transferase or a hexahistidine tag to an anti-his antibody or digoxigenin to antidigoxigenin, and appropriate target biomolecules can be determined accordingly.

The target biomolecule 150 can act as a linker group to which the biomolecule of interest 160 can bind. For example, in the case of streptavidin, which includes 4 monomers, each can bind with a biotin, the streptavidin can bind to both the functional molecule 125 on the bottom surface 122 and the biomolecule of interest 160. Alternatively, target molecule 150 can itself be fluorophore-labeled and become a subject of fluorescence study using the ZMW.

In some embodiments, in order to observe or study single molecule fluorescence imaging using the ZMW, the second functional molecule can include a mixture of (1) a molecule comprising polyethylene glycol and having a moiety capable of binding with a target biomolecule (illustrated in FIG. 1 by the slightly larger molecule having a triangle-shaped moiety 126); and (2) a molecule comprising polyethylene glycol and having no moiety capable of binding with the target biomolecule (illustrated in FIG. 1 by the remaining molecules). By controlling the ratio of the (1) type molecule and (2) type molecule, e.g., selecting the ratio to be sufficiently small, the nano-well can contain a single (or 2, 3, or other desired number of) target biomolecule 150 tethered onto the bottom surface via affinity binding, hence a single biomolecule of interest 160 tethered in the detection volume of the nano-well of the ZMW. This is illustrated by FIG. 2B, which shows silanization of the bottom surface of the nano-well 101 using two different molecules, silane-PEG, and silane-PEG-biotin. The molecular weights of the molecules shown in FIG. 2B are provided only as an example and not limiting.

In another aspect, the disclosed subject matter provides a method for fabricating the ZMWs as described above. In the method, a nano-well having a bottom surface and a gold side wall can be formed on a substrate, as will be further explained below. The dimensions and other characteristics of the nano-well have been described above. A surface of the side wall can be passivated with a first functional molecule comprising polyethylene glycol. The first functional molecule can include a thiol end group, and in such a case, the passivating can be accomplished by incubating the first functional molecule with the ZMW to form a S—Au bond coupling the first functional molecule with the gold surface.

The fabrication method can further include functionalizing the bottom surface of the nano-well with at least one second functional molecule comprising polyethylene glycol. The second functional molecule can include a silane end group, and in such a case, the functionalizing can be accomplished by reacting the silane end group with the bottom surface to form a Si—O—Si bond coupling the second functional molecule with the bottom surface. The at least one second molecule can also include a mixture of silane-PEG and silane-PEG-moiety, where the moiety is capable of binding with a target biomolecule, as discussed above and in connection with FIGS. 1 and 2. The moiety can be a biotin moiety, and in such case, the target biomolecule can be streptavidin.

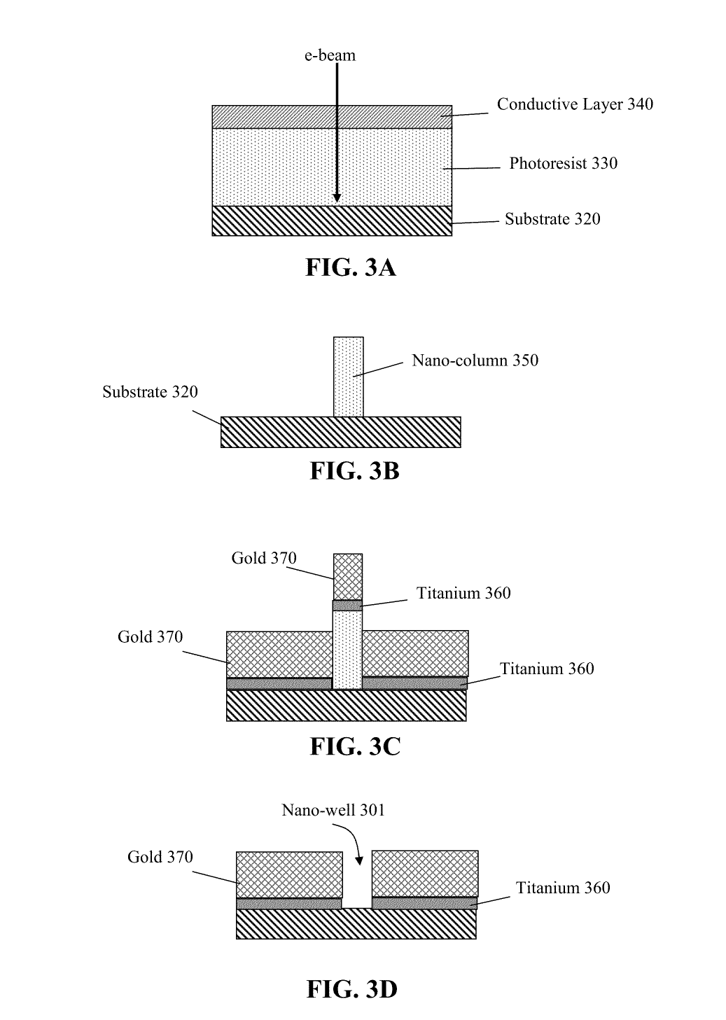

In one embodiment, forming the nano-well in the above method includes a procedure illustrated by the diagrams shown in FIGS. 3A-3D. A photoresist 330 (e.g., a negative resist) can be applied on the surface of the silica substrate 320, and a conductive layer 340, such as a conductive polymer, can be applied on top of photoresist layer 330 (FIG. 3A). Etching the structure, e.g., by electron beam lithography, can produce a nano-column 350 (FIG. 3B). A thin layer of titanium 360 (or another metal, such as chromium) can be deposited on the substrate 320 and the nano-column 350, and further, a layer of gold 370 can be deposited onto the layer of titanium 360 (FIG. 3C). The deposition of the metals can be by chemical vapor deposition or electron beam evaporation deposition, or other techniques known in the art. The nano-column 350, along with the titanium and gold layer deposited on top of it, can be removed, e.g., by sonication, thereby creating the nano-well 301.

Further details of the structure, fabrication, and use of the above-described ZMWs can be found in the following Examples, which are provided for illustration purpose only and not for limitation.

### Example 1

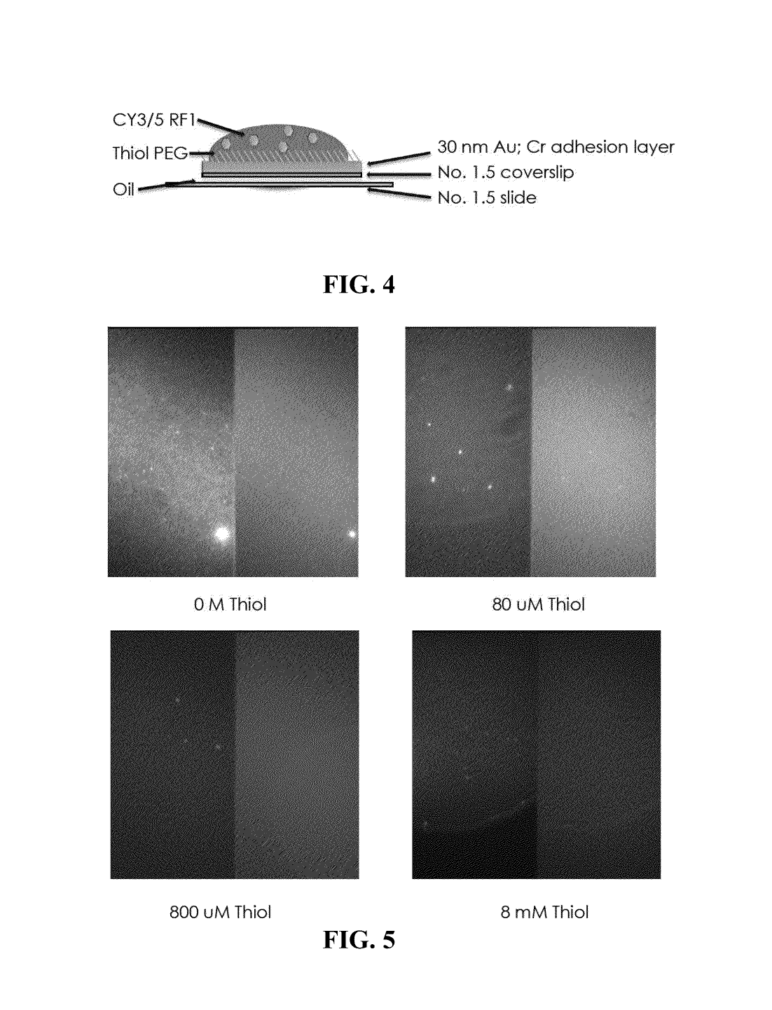

As with some of the other Examples below, Fluorescently labeled release factor 1 (RF1) which catalyzes nascent polypeptide chain release during the termination stage of protein synthesis by the ribosome, was used as a fluorophore (Cy3 and Cy5)-labeled test biomolecule of interest. All cysteine residues native to RF 1 were mutated to serine (C51S, C201S, C257S), and two cysteine residues were introduced at positions of a distance of approximately 40 Å apart (S192C, E256C), all using site-directed mutagenesis. These two cysteine residues were labeled with Cy3- and Cy5-maleimides at the reactive sulfhydryls groups, and purified using fast protein liquid chromatography (FPLC). A biotin molecule was covalently attached to the protein with a biotin ligase. FIG. 4 shows a schematic of the test. A solution containing RF1 was placed upon an optically transparent layer of gold that was passivated with thiolated PEG. This surface was imaged with a single molecule fluorescence microscope in epi-fluorescence mode. FIG. 5 show images captured with the microscope of gold surfaces treated with various concentrations of thiol-PEG to form SAMs. It can be seen that even at concentrations greater than 80 μM, non-specific binding appears negligible, as indicated by the significant decrease in fluorescence intensity on both the left-hand side (Cy3 fluorescence intensity) and the right-hand side (Cy5 fluorescence intensity) of the images.

### Example 2

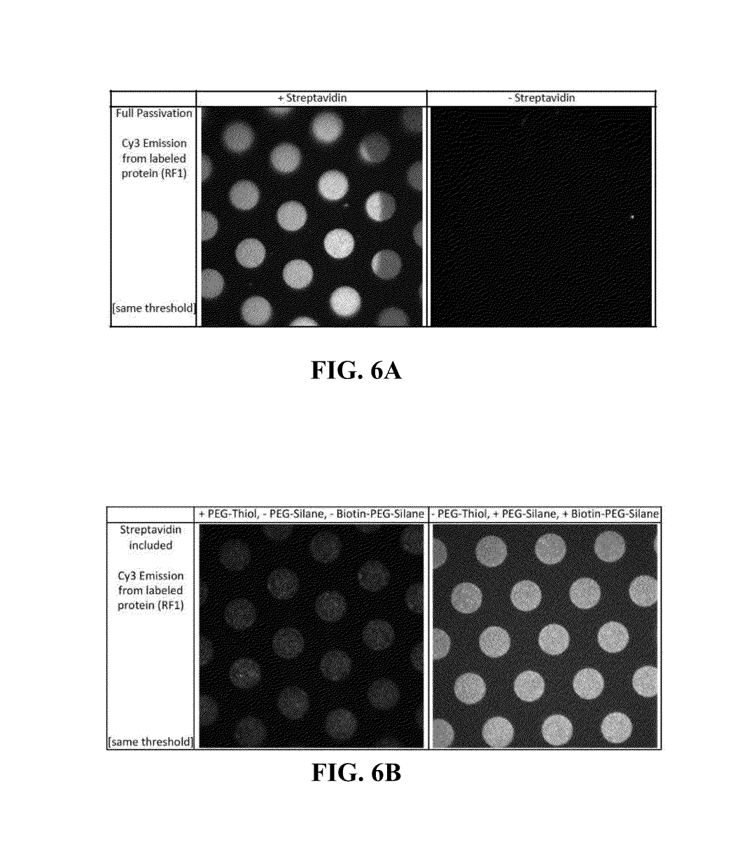

A glass slide was coated with a 100 nm thick gold, and a plurality of circular, micro-sized wells of 5 μm in diameter were made on the gold layer to expose the silica surface. As in Example 1, RF1 labeled with Cy3 and Cy5 was used as the test biomolecule. For the results shown in FIG. 6A, a thiol-PEG molecule was used to passivate the gold surface, and then a dilute solution of silane-PEG-biotin (in its mixture with PEG-silane) was used to passivate the exposed silica areas. As shown in FIG. 6A, when streptavidin was used (left image), the fluorescence signals of the RF 1 were prominent (Cy3 emission, as indicated by the bright circular areas). As a control test, FIG. 6B shows that when the gold was not passivated whereas the silica was passivated by silane-PEG/biotin-PEG-silane, the fluorescence of RF1 was diminished by only a few percent. This can be explained by the fact that streptavidin absorbs on bare gold more than RF 1. In contrast, when only the gold surface was passivated by thiol-PEG and the silica surface was not passivated, the Cy3 emission of the RF1 was significantly reduced.

### Example 3

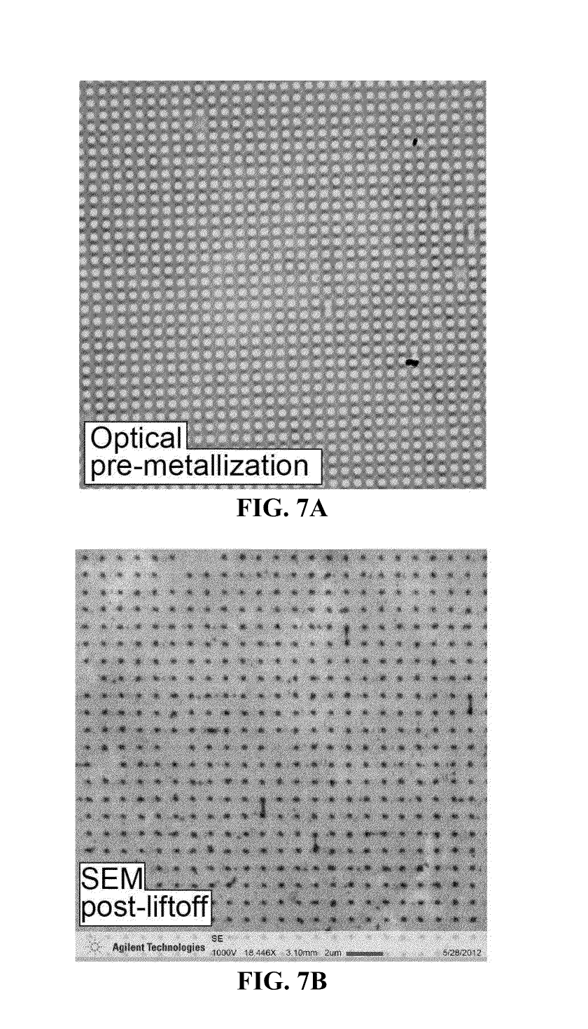

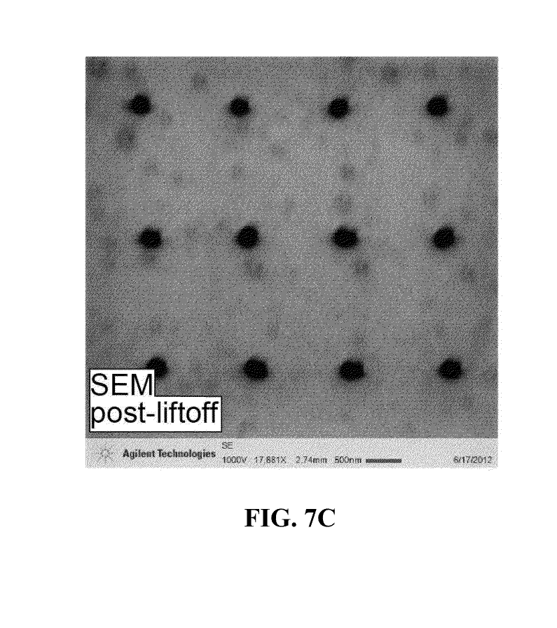

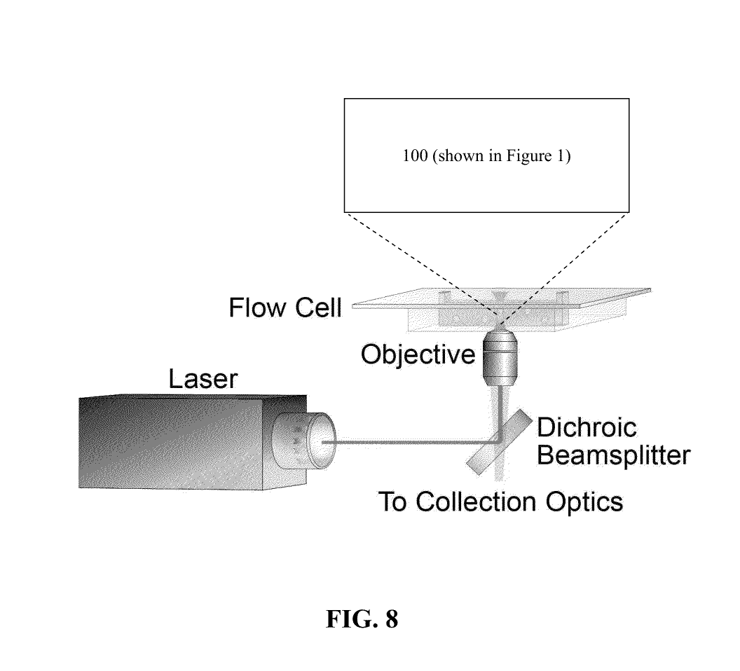

Gold-based ZMWs having aperture diameters ranging between 200-250 nm were fabricated as follows. (The general procedure of the fabrication has been schematically shown in FIG. 2.) After cleaning No. 1½ glass coverslips with successive sonication, flaming, piranha etching, and oxygen plasma processing, a thin layer of negative-tone resist, Ma-N 2403, was deposited with a spin coater onto the coverslips. The structure obtained was then prebaked at 90° C., and a highly conductive polymer, PEDOT:PSS 2.2% in H₂O, was deposited and then prebaked again at 90° C. Electron beam lithography was performed with a converted FEI Sirion SEM, and was employed to pattern arrays of circles of diameters on the order of about 100 nm using the Nanometer Pattern Generation System (JC Nabity Lithography Systems). Electrons from the electron-beam gun crosslinked the negative-tone resist and excess charge was dissipated to a ground by the conductive layer. The patterns were developed, with non-crosslinked photoresist removed, leaving behind cylindrical columns. An optical micrograph of the patterns is shown in FIG. 7A. Atop these columns, an optically transparent layer of 1 nm of titanium was deposited with an electron beam gun using an Angstrom EvoVac Deposition System to increase the adhesion of gold to the substrate. Approximately 100 nm of gold was then deposited in a similar fashion, such that the metallization process did not cover the entire height of the patterned columns, leaving the columns exposed to solvent. Finally, sonication in extremely basic, aqueous solution induced liftoff of the columns, removing residual photoresist and forming ZMWs in the relief Approximately 78% of the fabricated patterns were tested to be functional ZMWs with nano-well diameter of about 200±15 nm (1σ), characterized with an Agilent 8500 FE-SEM and atomic force microscopy. SEM micrographs of the fabricated ZMWs including arrays of nano-wells are shown in FIG. 7B, and FIG. 7C (which is an enlarged image of a portion of FIG. 7B).

### Example 4

The ZMWs fabricated by Example 3 was passivated to reduce non-specific adsorption. The passivation procedure started with cleaning the ZMWs in aged piranha solution, followed by a short treatment by oxygen plasma. The cleaned ZMWs were incubated in 5 mM anhydrous ethanolic solutions of PEG-SH (MW=350 g/mol) (Nanocs, Boston, Mass.) for 12 hours to thiolate the gold surfaces, rinsed thoroughly in EtOH, and dried with N₂. Silanization was performed by mixing a predetermined molar ratio of biotin-PEG-Si—(OCH₃)₃ (MW=3400 g/mol) to mPEG-Si—(OCH₃)₃ (MW=2000 g/mol) (Laysan Bio Inc., Arab, Ala.) (as shown in FIGS. 2A and 2B) in anhydrous toluene, with a catalytic amount of glacial acetic acid, such that the total concentration of silane was on the order of 100 μM. The ZMWs were incubated in the silane solution for 24 hours, rinsed with distilled, deionized water for 15 minutes, rinsed with ethanol, and blown dry with N₂.

### Example 5

Passivated ZMWs were used in fluorescence imaging of a biomolecule. A schematic setup of the fluorescence measurement is shown in FIG. 8. Samples of biomolecule of interst were epi-illuminated with a 532 nm diode-pumped laser (Crystal Laser) on a Nikon Ti-U microscope with a 552 nm, single-edge dichroic beamsplitter (Semrock) and a 533 nm (FWHM=17 nm) notch filter (Thorlabs) in a filter cube through a Nikon, water-immersion 60X, NA=1.2 Plan Apo objective. Fluorescence was collected through a Photometrics DV2 with a 630 dcxr dichroic beamsplitter, and HQ575/40m and HQ680/50m emission filters upon an Andor iXon3 897E.

RF1 labeled with Cy3 and Cy5 fluorophores within FRETing distance as described above were anchored to the bottom of the nano-wells of the ZMWs prepared according to the above-described procedure via conjugation to a streptavidin molecule which had previously been conjugated to a biotin at the bottom of each ZMW. After washing, an oxygen scavenging system (GOD/CAT) and a triplet quenching system were washed in for imaging. For some tests, this imaging buffer also included fluorophore-labeled biomolecules. 2000 frame movies were collected using Metamorph (Molecular Devices) with a 100 ms acquisition rate, 14-bit ADC, 10 MHz horizontal shift, 3.33 MHZ vertical shift, and a linear EM gain of 200. These were analyzed with homegrown python scripts. Spots were chosen after thresholding a background corrected image to three standard deviations above the mean intensity in the Cy3 channel of the DV2. These coordinates were monitored in the Cy3 channel and translated into their corresponding spot on the Cy5 channel by tracking the center of mass of the entire image to correct for drift, and applying this correction to the location of the particle spot of interest at each frame. Intensities were summed area-dependently upon the neighboring four pixels, such that the total spot area was one pixel. Single-step photobleaching events were located by convoluting the signal with a function reminiscent of a negative, odd-valued (v=1) harmonic oscillator wavefunetion, thresholding this to +3σ, and then locating local maxima.

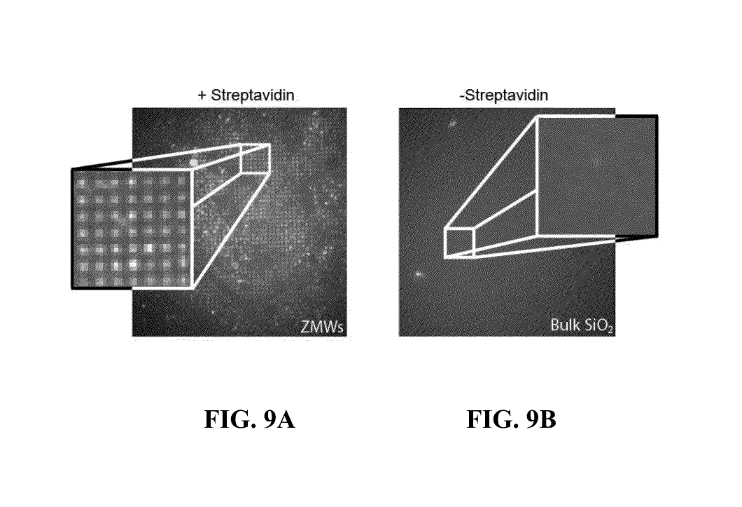

As shown in FIG. 9, in the presence of streptavidin (left), single-molecule fluorescence is observable originating from the ZMWs. Without streptavidin (right), the thiol and silane passivated surfaces of the ZMW resist non-specific binding of this protein entirely. This indicates that thiol- and silane-based SAM passivation of the side gold wall and bottom silica surface substantially prevents non-specific binding of RF1(Cy3,Cy5).

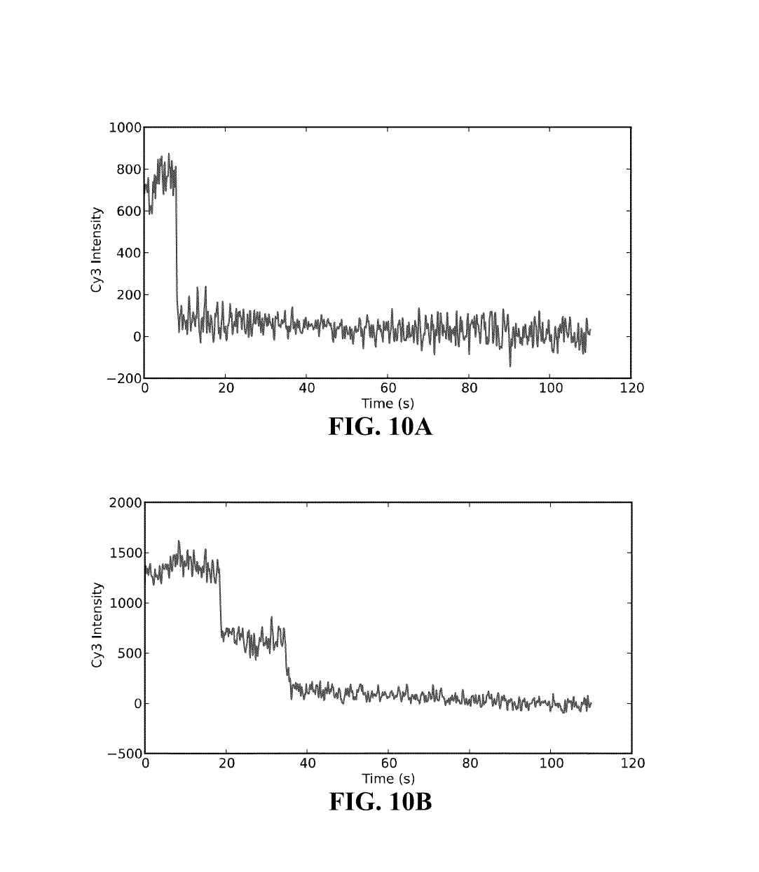

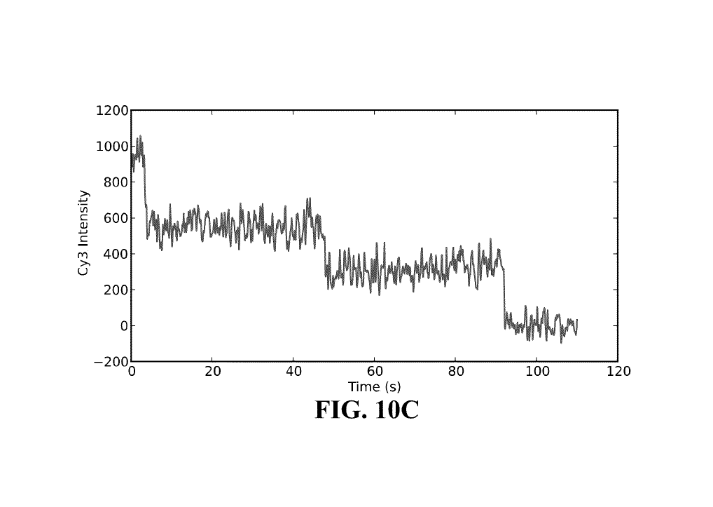

As shown in FIGS. 10A-10C, the ratio of silane-PEG to biotinylated silane-PEG can be used to control the average number of biotinylated, RF1(Cy3,Cy5)s that are tethered to the bottom of individual nano-wells of the ZMWs. Single-step photobleaching was used as a proxy for the presence of a single-molecule within a ZMW. Based on the fluorescence emitted from different nano-wells, 1, 2, and 3 single-step photobleaching events were identified, shown in FIGS. 10A, 10B, and 10C, respectively, indicating the presence of 1, 2, and 3 single-molecules within each respective nano-well of the ZMWs.

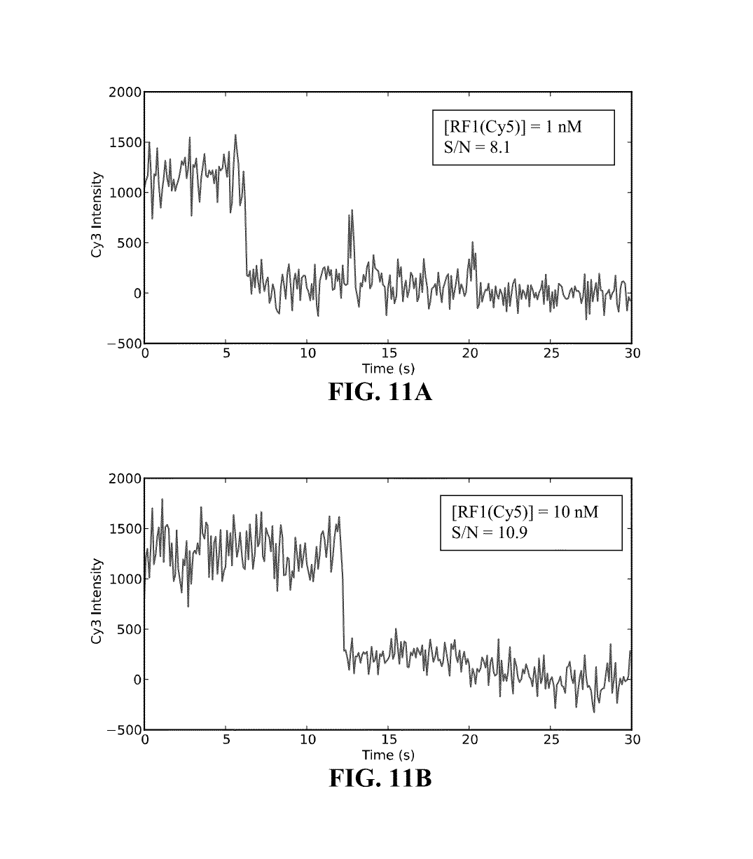

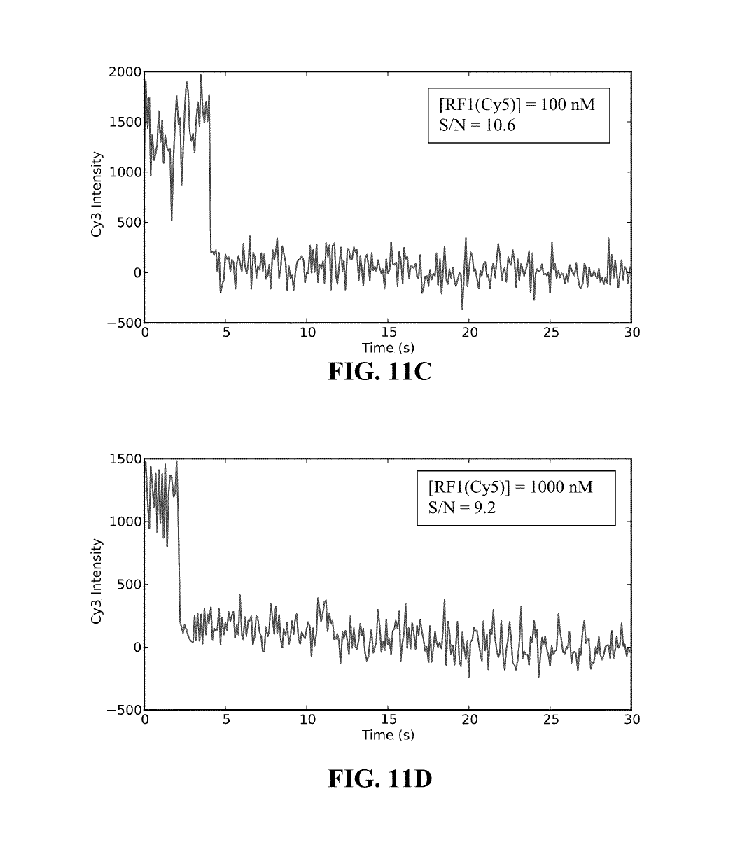

High signal-to-noise ratios can be achieved using the disclosed ZMWs despite high background concentrations of RF1(Cy5). As shown in FIGS. 11A-11D, signal-to-noise ratio of the fluorescence signal of a single-molecule within a passivated ZMW (both the gold surface and the silica surface) does not decrease significantly with the titration of fluorophore-labeled protein into the ZMWs over a range that includes 0, 1, 10, 100, and 1000 nanomolar concentrations of this protein. Reaching 1000 nanomolar concentrations of background, fluorophore-labeled protein can allow many systems to be investigated with the disclosed ZMWs by fluorescence microscopies at physiologically-relevant concentrations of fluorophore-labeled biomolecules.

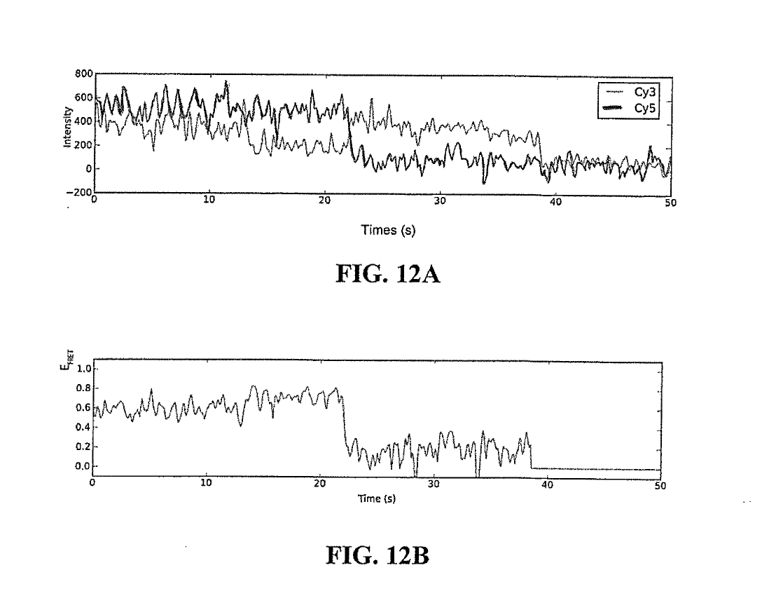

Further, intramolecular Fluorescence Resonance Energy Transfer (FRET) originating from individual RF1(Cy3,Cy5) molecules can be observed and investigated using the disclosed gold-based, passivated ZMWs, as illustrated in FIGS. 12A and 12B. The fluorescence intensity time traces of Cy3 and Cy5 originating from a single molecule of RF1(Cy3,Cy5) shown in FIG. 12A can be collapsed into a single signal representing the FRET efficiency by normalizing the Cy5 fluorescence to the total fluorescence signal recorded, which can be plotted as a function of time as shown in FIG. 12B. The single-step photobleaching event observed in the FRET efficiency is an unambiguous proxy for the presence of a single, FRETing molecule of RF1(Cy3,Cy5) within a ZMW.

While the disclosed subject matter is described herein in terms of certain embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having other combinations of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A zero-mode waveguide, comprising:
a substrate,
at least one nano-well on the substrate, the at least one nano-well having a bottom surface and a side wall comprising gold;
wherein a surface of the side wall is passivated with a first functional molecule comprising polyethylene glycol.

2. The zero-mode waveguide of claim 1, wherein the at least one nano-well has a width of from about 25 to about 500 nm.

3. The zero-mode waveguide of claim 1, wherein the side wall has a height of from about 50 to about 500 nm.

4. The zero-mode waveguide of claim 1, wherein the first functional molecule is attached to the surface of the side wall via a S—Au bond.

5. The zero-mode waveguide of claim 1, wherein the first functional molecule form a monolayer on the side wall.

6. The zero-mode waveguide of claim 1, wherein the first functional molecule further comprises polyalkylene.

7. The zero-mode waveguide of claim 6, wherein the polyethylene glycol of the first functional molecule comprises from about 1 to about 200 ethylene oxide units.

8. The zero-mode waveguide of claim 1, wherein the bottom surface of the at least one nano-well is functionalized with at least one second molecule comprising polyethylene glycol.

9. The zero-mode waveguide of claim 8, wherein the polyethylene glycol of the second functional molecule comprises from about 1 to about 200 ethylene oxide units.

10. The zero-mode waveguide of claim 8, wherein the at least one second functional molecule comprises a molecule having a moiety capable of binding with a target biomolecule.

11. The zero-mode waveguide of claim 10, wherein the moiety comprises a biotin moiety.

12. The zero-mode waveguide of claim 11, wherein the target biomolecule is streptavidin.

13. The zero-mode waveguide of claim 8, wherein the bottom surface comprises silica, and the at least one second functional molecule is attached to the bottom surface via a Si—O—Si linkage.

14. The zero-mode waveguide of claim 8, wherein the at least one second functional molecule comprises a mixture of (1) a molecule comprising polyethylene glycol and having a moiety capable of binding with a target biomolecule, and (2) a molecule comprising polyethylene glycol and having no moiety capable of binding with the target biomolecule.

15. The zero-mode waveguide of claim 13, further comprising the target biomolecule bound to the moiety.

16. The zero-mode waveguide of claim 1, further comprising a layer of titanium or chromium disposed between the substrate and the side wall of the at least one nano-well.

17. A method for fabricating a zero-mode waveguide, comprising:
forming at least one nano-well on a substrate, the nano-well having a bottom surface, and a side wall comprising gold; and
passivating a surface of the side wall with a first functional molecule comprising polyethylene glycol.

18. The method of claim 17, wherein the first functional molecule comprises a thiol end group, and wherein the passivating comprises reacting the thiol end group with the surface of the side wall to form a S—Au bond coupling the first functional molecule with the surface.

19. The method of claim 17, further comprising functionalizing the bottom surface of the at least one nano-well with at least one second molecule comprising polyethylene glycol.

20. The method of claim 19, wherein the at least one second functional molecule comprises a silane end group, wherein the bottom surface of the at least one nano-well comprise silica, and wherein the functionalizing comprises reacting the silane end group with the bottom surface to form a Si—O—Si bond coupling the second functional molecule with the bottom surface.

21. The method of claim 19, wherein the functionalizing comprises functionalizing the bottom surface with a mixture of (1) a molecule comprising polyethylene glycol and having a moiety capable of binding with a target biomolecule, and (2) a molecule comprising polyethylene glycol and having no moiety capable of binding with the target biomolecule.

22. The method of claim 21, wherein the moiety comprises a biotin moiety, and the target biomolecule is streptavidin.

23. The method of claim 17, wherein the substrate is a silica substrate, wherein the forming further comprises:
applying a photoresist on the surface of the silica substrate;
forming at least one nano-column in the photoresist by etching;
depositing a thin layer of titanium on the substrate;
depositing a layer of gold onto the layer of titanium; and
removing the at least one nano-column in the photoresist, thereby creating the at least one nano-well having a bottom surface and a side wall comprising gold.
